(19) United States
(12) Patent Application Publication
(10) Pub. No.: US 2020/0234790 A1
(43) Pub. Date: Jul. 23, 2020

(54) DNA REPAIR PROFILING AND METHODS THEREFOR

(71) Applicant: NANT HOLDINGS IP, LLC, Culver City, CA (US)

(72) Inventors: Patrick SOON-SHIONG, Culver City, CA (US); Shahrooz RABIZADEH, Culver City, CA (US); Kayvan NIAZI, Culver City, CA (US)

(21) Appl. No.: 16/637,235

(22) PCT Filed: Aug. 7, 2018
(86) PCT No.: PCT/US2018/045654

§ 371 Date: Feb. 6, 2020

Related U.S. Application Data
(60) Provisional application No. 62/542,281, filed on Aug. 7, 2017.

(51) Int. Cl. G16B 20/00; C12Q 1/6869; G16H 50/20; G16H 50/30; G16H 20/10

(52) U.S. Cl. CPC G16B 20/00; C12Q 1/6869; G16H 20/10; G16H 50/30; G16H 50/20

(57) ABSTRACT

Systems and methods are contemplated that use various omics data for DNA repair genes to assess a health associated parameter for an individual.

This application claims priority to our copending U.S. provisional application with the Ser. No. 62/542,281, which was filed Aug. 7, 2017.

### FIELD OF THE INVENTION

The field of the invention is profiling of omics data as they relate to DNA repair, and especially as it relates to the generation of a global health indicator, and to prophylactic and therapeutic methods and compositions to counteract age-related conditions and diseases.

### BACKGROUND OF THE INVENTION

The following description includes information that may be useful in understanding the present invention. It is not an admission that any of the information provided herein is prior art or relevant to the presently claimed invention, or that any publication specifically or implicitly referenced is prior art.

In addition to the inherent error-prone nature of various DNA polymerases, mammalian DNA is constantly subjected to chemical, physical, and metabolic challenges that can introduce chemical changes, loss of nucleobases, and DNA single and double strand breaks. Indeed, it is estimated that each of the approximately 10¹³ cells within the human body incurs tens of thousands of DNA-damaging events per day (see e.g., Lindahl T, Barnes DE (2000) Repair of endogenous DNA damage. Cold Spring Harb Symp Quant Biol 65:127-133). All publications and patent applications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

While such damage often results in genomic instability and cell death, many of these lesions also cause structural damage to DNA and can alter or eliminate fundamental cellular processes, such as DNA replication or transcription. To counteract the harmful effects of DNA damage, cells have various DNA repair systems, including base excision repair, mismatch repair, nucleotide excision repair, and double-strand break repair, which comprise both homologous recombination and non-homologous end-joining.

Previous experimental data on animals having defects in DNA repair genes often showed a decreased life span and increased cancer incidence. For example, mice that were deficient in the dominant NHEJ (non-homologous end-joining) pathway and in telomere maintenance mechanisms were prone to lymphoma and infections, and typically had shorter lifespans than wild-type mice. In a similar manner, mice that were deficient in a key repair and transcription protein that unwinds DNA helices had often premature onset of age-related diseases and shortening of lifespan. However, the effects of deficiencies in DNA repair are not readily predictable: mice having a deficient NER pathway tend to exhibit shortened life span without correspondingly higher rates of mutation. With further respect to cancer, various known DNA repair gene mutations are associated with increased cancer risk. For example, hereditary nonpolyposis colorectal cancer (HNPCC) is strongly associated with specific mutations in the DNA mismatch repair pathway, while BRCA1 and BRCA2 are associated in breast cancer with a large number of DNA repair pathways, especially NHEJ and homologous recombination. More recently, mutations in DNA repair genes were also implicated in cancer metastases (see e.g., Radiation Research 181, 111-130 (2014)). However, no discernible pattern exists for DNA repair genes that could be used to predict the effect of an increased or decreased activity of a particular DNA repair pathway.

Therefore, while numerous experimental details are known for DNA repair genes and pathways, there is a lack of systemic understanding and use of DNA repair genes and pathways in the assessment of health and treatment recommendations.

### SUMMARY OF THE INVENTION

The inventive subject matter provides systems and methods in which multiple omics data for various DNA repair genes of a patient sample are employed to derive one or more health associated parameter. For example, preferred omics data include DNA sequence data, RNA sequence data, and particularly transcription strength, and/or protein activity or protein quantity, while especially preferred health associated parameters include health status, error status, and treatment recommendations. Moreover, expression levels (transcription strength) of various DNA repair genes can be used to assess real-time status of the DNA repair system to indicate overall health, presence and/or severity of DNA damage (due to environmental factors or pharmaceutical intervention), and as such can be used to monitor response to a treatment or to predict recurrence of disease.

In one aspect of the inventive subject matter, the inventors contemplate a method of analyzing omics data that includes a step of obtaining omics data for a plurality of DNA damage repair genes, wherein the omics data comprise at least two of DNA sequence data, RNA sequence data, transcription strength, and protein activity or quantity. In another step, the omics data are then associated with a health status, an omics error status, age, a disease, a prophylactic recommendation, and/or a therapeutic recommendation. Where desired, contemplated methods may further include a step of calculating a score from the omics data to so obtain a health score.

With respect to DNA sequence data it is contemplated that such data may include mutation data, copy number data duplication, loss of heterozygosity data, and/or epigenetic status, while RNA sequence data may include mRNA sequence data and splice variant data, which may be obtained from solid tissue, from blood cells, and/or from circulating cell free RNA. Moreover, it is generally preferred that the transcription strength is expressed as transcripts of the damage repair gene per million transcripts, and/or that protein activity or quantity is determined using a mass spectroscopic method (e.g., using a selective reaction monitoring method).

The health status may typically include a healthy status, a diagnosis with an age related disease, and a diagnosis with cancer. Contemplated prophylactic recommendation will include a recommendation to treat an individual with an agent that modulates expression of at least one of the plurality of DNA damage repair genes, while therapeutic recommendations may comprise a recommendation to treat a patient with a DNA damaging agent. Suitable DNA damage repair genes will include one or more of a base excision repair gene, a mismatch repair gene, a nucleotide excision repair gene, a homologous recombination gene, and a non-homologous end-joining gene, and exemplary DNA damage repair genes are listed in Tables 1-3 below.

Contemplated steps of associating the omics data with a status may comprise a weight score for at least one of the omics data, and it is further contemplated that such method may further comprise a step of comparing the omics error status with a threshold value to thereby determine a risk score.

Therefore, and viewed from a different perspective, the inventors also contemplate a method of calculating a health indicator that includes a step of obtaining omics data for a plurality of DNA damage repair genes, wherein the omics data comprise at least two of DNA sequence data, RNA sequence data, transcription strength, and protein activity or quantity. The so determined omics data are then used to generate a health compound score that is indicative of the health of a person.

As noted above, contemplated methods may further comprise a step of comparing the compound score with a threshold value to thereby determine a treatment option. For example, the treatment option may be a prophylactic treatment where the compound score is below the threshold value, the treatment option may use a drug that modulates expression of at least one of the plurality of DNA damage repair genes, or the treatment option may use a drug that induces DNA damage.

In yet another aspect of the inventive subject matter, the inventors also contemplate a method of treating an individual that includes the steps of obtaining omics data for a plurality of DNA damage repair genes, wherein the omics data comprise at least two of DNA sequence data, RNA sequence data, transcription strength, and protein activity or quantity, and a further step of identifying at least one of the DNA damage repair genes as being dysregulated relative to a corresponding healthy control. In yet another step, an agent is then administered that counteracts the at least one of the dysregulated DNA damage repair gene.

Most typically, DNA sequence data are selected from the group consisting of mutation data, copy number data duplication, loss of heterozygosity data, and epigenetic status, while the RNA sequence data are selected from the group consisting of mRNA sequence data and splice variant data. As noted the RNA sequence data may be obtained from solid tissue, blood cells, and/or circulating cell free RNA. Most typically, the transcription strength is expressed as transcripts of the damage repair gene per million transcripts, and/or the protein activity or quantity is determined using a mass spectroscopic method. With respect to the DNA damage repair genes it is contemplated that the at least one or more of the DNA damage repair genes a base excision repair gene, a mismatch repair gene, a nucleotide excision repair gene, a homologous recombination gene, and/or a non-homologous end-joining gene. For example, suitable DNA damage repair genes are listed in Table 1, Table 2, and Table 3.

Therefore, the inventors also contemplate a method of performing a test on a subject that includes a step of obtaining a blood sample from the subject, and another step of using the blood sample to obtain omics data for a plurality of DNA damage repair genes, wherein the omics data comprise at least two of DNA sequence data, RNA sequence data, transcription strength, and protein activity or quantity. Most preferably, the omics data are obtained from a cell free portion of the blood sample and/or a cell containing portion of the blood sample. In still another step of contemplated methods, at least one of the DNA damage repair genes is identified in the blood sample as being dysregulated relative to a corresponding healthy control.

Most typically, the RNA sequence data are selected from the group consisting of mRNA sequence data and splice variant data, and the RNA sequence data may be obtained from solid tissue, from blood cells, and/or circulating cell free RNA. The transcription strength is preferably expressed as transcripts of the damage repair gene per million transcripts. As noted above, preferred DNA damage repair genes are selected from a base excision repair gene, a mismatch repair gene, a nucleotide excision repair gene, a homologous recombination gene, and a non-homologous end-joining gene. For example, exemplary DNA damage repair genes include those listed in Table 1, Table 2, and Table 3.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary illustration of various modes of DNA damage, resultant lesions, and repair pathways to counteract such damage.

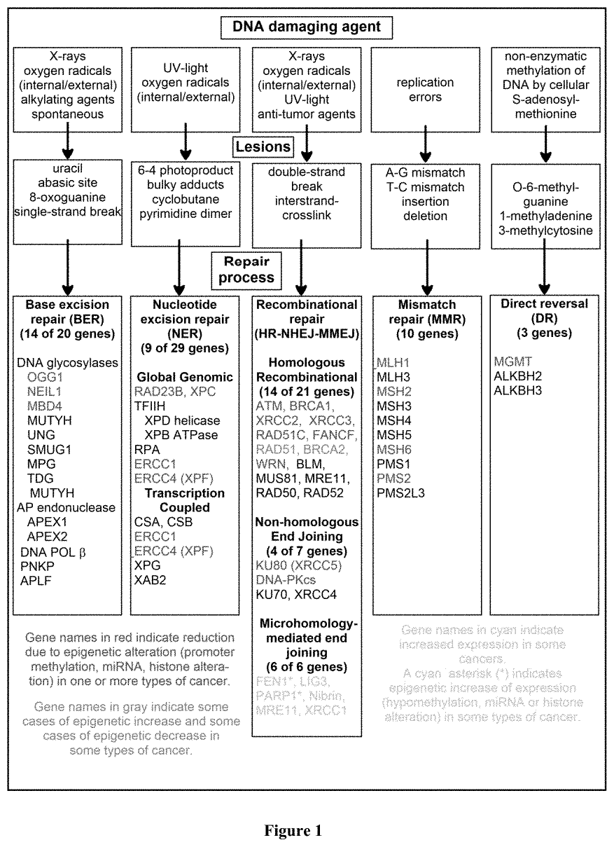

### DETAILED DESCRIPTION

The inventors have now discovered that a library or reference database for all DNA repair genes can be created using one or more omics data for each gene associated with DNA repair, and that such library is particularly useful where the omics data are associated with one or more health parameter. Such library or reference database may be particularly useful where expression levels of DNA damage repair genes are quantified and/or where mutations (and particularly mutations affecting DNA repair) are detected, and where such quantities and detected mutations are associated with a particular health status.

Viewed from a different perspective, the inventors contemplate that signatures for omics data from DNA repair associated genes can be identified that are characteristic for the error status within a patient, which in turn may be indicative for one or more health related conditions. Likewise, such signatures may be predictive of DNA damage even before the actual damage can be observed in a diseased tissue. As will be readily appreciated, signatures may be ascertained once (e.g., during a routine visit before signs or symptoms of a disease are evidence), or be followed over time for a single patient, which may be especially useful where health is generally assessed, or where a disease or treatment is monitored.

While traditional studies of DNA repair have often focused on the presence or strength of expression of a particular gene associated within a single DNA repair pathway, the inventors now contemplate that such analysis is insufficient to obtain an indicator that has predictive or even analytic power with respect to a health condition or likely treatment outcome. To that end, the inventors have discovered that DNA repair genes can be analyzed not only as present or absent, but that a full scale omics analysis will take into account multiple aspects of multiple genes. More specifically, the inventors contemplate a library or reference database that catalogs not only DNA sequence data of DNA repair associated genes, but also corresponding RNA sequence data, corresponding transcription strength, and corresponding protein activity and/or quantity of multiple DNA repair associated genes to so provide a dynamic picture of DNA repair activity.

Advantageously, and particularly where contemplated signatures for DNA repair associated genes (e.g., expression levels of one or more DNA repair associated genes) are combined with omics data from diseased tissue, mutational patterns in diseased tissue can be correlated with the signatures for confirmation of treatment as well as prediction of treatment outcome. Moreover, where contemplated signatures for DNA repair associated genes include analyses for gene damage in the DNA repair associated genes, such mutational damage may be predictive for hypermutations in the tumor genome due to lack of an efficient repair system.

Therefore, DNA sequence data will not only include the presence or absence of a gene that is associated with DNA repair, but also take into account mutation data where the repair associated gene is mutated, the copy number (e.g., to identify duplication, loss of allele or heterozygosity), and even epigenetic status (e.g., methylation, histone phosphorylation, nucleosome positioning, etc.). With respect to RNA sequence data it should be noted that contemplated RNA sequence data include mRNA sequence data, splice variant data, polyadenylation information, etc. Moreover, it is generally preferred that the RNA sequence data also include a metric for the transcription strength (e.g., number of transcripts of a damage repair gene per million total transcripts, number of transcripts of a damage repair gene per number of transcripts for actin or other household gene RNA, etc.). It should be noted that such transcription strength information is particularly useful where transcription strength is measured over time to detect an increase in a particular type of DNA damage. Similarly, it is generally preferred that contemplated analyses may also include one or more metrics that can quantify protein activity and/or protein quantity for a particular gene associated with DNA repair. For example, suitable protein activity or quantity can be determined using known enzymatic assays, and/or various mass spectroscopic methods, and especially selected reaction monitoring methods such as multiple reaction monitoring and parallel reaction monitoring.

Of course, it should be noted that the omics data can be obtained in numerous manners and from numerous sources, and especially preferred source materials include whole blood and cell-containing and cell-free portions thereof, and tissue biopsies from diseased and/or healthy organs of an individual. For example, DNA and RNA may be obtained from solid tissue, from blood cells, and/or from a pool of circulating cell free RNA. In other examples, DNA, RNA, and/or protein may be obtained from a tissue biopsy (e.g., fresh, frozen, or FFPE), which may be collected together with a sample of corresponding healthy tissue. In further preferred aspects, omics data can also be obtained from single cell sequencing. Moreover and as already noted earlier, the omics data can be obtained from more than one tissue or source, and over multiple points in time. For example, omics data may be initially obtained from biopsy material of a diseased tissue and a further non-diseased sample of the same patient (e.g., skin, blood, etc.). Alternatively, or additionally, omics data may be initially obtained from circulating nucleic acids (and especially cfRNA (circulating cell free RNA)) of a blood draw or other biological fluid, alone or in combination with omics data from healthy and/or diseased tissue.

Advantageously, it should be noted that the omics data can also be obtained at a point in time prior to a treatment (or even a diagnosis), during treatment, and/or after a treatment. Similarly, omics data can be obtained prior to or after exposure to a particular environment (e.g., prior to entry into a chemically or radiologically contaminated area), or prior to or after exposure to a particular DNA damaging condition (e.g., sun exposure, RF exposure, etc.). As will be readily appreciated, repeated acquisition of omics data will allow identification of trends in triggering or maintenance of a DNA damage response, which in turn specifically indicates the type and severity of cellular stress.

In addition, it is contemplated that the omics data for the genes associated with DNA repair may be acquired in parallel (or at some other time) with omics data for non-repair relevant genes that are specific for a diseased tissue. For example, conventional nucleic acid analysis will typically only identify mutations of a tumor tissue relative to normal tissue. In contrast, contemplated analyses may include omics data for genes associated with DNA repair together with omics data of non-repair relevant genes specific for a diseased tissue (tumor specific mutations or tumor specific changes in gene expression). Such analysis is especially useful where the omics data for the non-repair relevant genes are used in pathway analysis as such combined data will not only allow identification of activity and status of genes associated with DNA repair, but also physiological activity that is relevant in the context of DNA repair. For example, pathway analysis may reveal that certain pathways (e.g., apoptosis or other cell death relevant pathway) are activated where activity of genes associated with DNA repair is increased, which may be indicative of a treatment success. On the other hand, other pathways may be activated (e.g., pathways associated with EMT) where activity of genes associated with DNA repair is increased, which may be indicative of potential treatment failure. Therefore, contemplated combined analyses will add further functional information of a cell in the context of cell stress and DNA repair.

As should also be readily appreciated, the type of omics data will vary considerably and will typically depend on the type of sample used, omics parameter (e.g., genomic data, transcriptomic data, proteomic data, etc.), and/or desired omics data characteristic (e.g., mutational information, strength of transcription, protein activity, pathway activity, etc.). Consequently, suitable omics data include as raw data (e.g., FASTQ), differential data (e.g., after BAMBAM analysis), various processed data (e.g., VCF format), or even as data after analysis using pathway analysis (e.g., using PARADIGM).

Therefore, and viewed from a different perspective, it should be appreciated that omics analysis across multiple genes associated with DNA repair the library will provide a detailed insight with respect to integrity and/or activity of DNA repair associated genes and pathways, and as such allows for a quantitative analysis of the overall mutation status of a genome, and more particularly of the mutation and functional status of the DNA repair mechanisms in a patient or other individual.

With respect to contemplated genes associated with DNA repair, Table 1 provides an exemplary collection of predominant DNA repair genes and their associated repair pathways presented herein, and a typical library of genes associated with DNA repair will include one, or two, or three, or four, or more of at least two repair categories of Table 1.

TABLE 1

| Repair mechanism | Predominant DNA Repair genes |
|---|---|
| Base excision repair (BER) | DNA glycosylase, APE1, XRCC1, PNKP, Tdp1, APTX, DNA |
| | polymerase β, FEN1, DNA polymerase δ or ε, PCNA-RFC, PARP |
| Mismatch repair (MMR) | MutSα (MSH2-MSH6), MutSβ (MSH2-MSH3), MutLα (MLH1- |
| | PMS2), MutLβ (MLH1-PMS2), MutLγ (MLH1-MLH3), Exo1, |
| | PCNA-RFC |
| | XPC-Rad23B-CEN2, UV-DDB (DDB1-XPE), CSA, CSB, TFIIH, |
| Nucleotide excision repair (NER) | XPB, XPD, XPA, RPA, XPG, ERCC1-XPF, DNA polymerase δ or ε |
| Homologous recombination (HR) | Mre11-Rad50-Nbs1, CtIP, RPA, Rad51, Rad52, BRCA1, BRCA2, |
| | Exo1, BLM-TopIIIα, GEN1-Yen1, Slx1-Slx4, Mus81/Eme1 |
| Non-homologous end-joining | Ku70-Ku80, DNA-PKc, XRCC4-DNA ligase IV, XLF |
| (NHEJ) | |

However, it should be recognized that numerous other genes associated with DNA repair and repair pathways are also expressly contemplated herein, and Tables 2 and 3 illustrate further exemplary genes for analysis and their associated function in DNA repair.

TABLE 2

| | | Accession |
|---|---|---|
| Gene name (synonyms) | Activity | number |
| Base excision repair (BER) | | |
| | DNA glycosylases: major altered base | |
| | released | |
| UNG | U excision | NM_003362 |
| SMUG1 | U excision | NM_014311 |
| MBD4 | U or T opposite G at CpG sequences | NM_003925 |
| TDG | U, T or ethenoC opposite G | NM_003211 |
| OGG1 | 8-oxoG opposite C | NM_002542 |
| MYH | A opposite 8-oxoG | NM_012222 |
| NTH1 | Ring-saturated or fragmented pyrimidines | NM_002528 |
| MPG | 3-meA, ethenoA, hypoxanthine | NM_002434 |
| | Other BER factors | |
| APE1 (HAP1, APEX, REF1) | AP endonuclease | NM_001641 |
| APE2 (APEXL2) | AP endonuclease | NM_014481 |
| LIG3 | Main ligation function | NM_013975 |
| XRCC1 | Main ligation function | NM_006297 |
| | Poly(ADP-ribose) polymerase (PARP) | |
| | enzymes | |
| ADPRT | Protects strand interruptions | NM_001618 |
| ADPRTL2 | PARP-like enzyme | NM_005485 |
| ADPRTL3 | PARP-like enzyme | AF085734 |
| Direct reversal of damage | | |
| MGMT | O6-meG alkyltransferase | NM_002412 |
| Mismatch excision repair (MMR) | | |
| MSH2 | Mismatch and loop recognition | NM_000251 |
| MSH3 | Mismatch and loop recognition | NM_002439 |
| MSH6 | Mismatch recognition | NM_000179 |
| MSH4 | MutS homolog specialized for meiosis | NM_002440 |
| MSH5 | MutS homolog specialized for meiosis | NM_002441 |
| PMS1 | Mitochondrial MutL homolog | NM_000534 |
| MLH1 | MutL homolog | NM_000249 |
| PMS2 | MutL homolog | NM_000535 |
| MLH3 | MutL homolog of unknown function | NM_014381 |
| PMS2L3 | MutL homolog of unknown function | D38437 |
| PMS2L4 | MutL homolog of unknown function | D38438 |
| Nucleotide excision repair (NER) | | |
| XPC | Binds damaged DNA as complex | NM_004628 |
| RAD23B (HR23B) | Binds damaged DNA as complex | NM_002874 |
| CETN2 | Binds damaged DNA as complex | NM_004344 |
| RAD23A (HR23A) | Substitutes for HR23B | NM_005053 |
| XPA | Binds damaged DNA in preincision | NM_000380 |
| | complex | |
| RPA1 | Binds DNA in preincision complex | NM_002945 |
| RPA2 | Binds DNA in preincision complex | NM_002946 |
| RPA3 | Binds DNA in preincision complex | NM_002947 |
| TFIIH | Catalyzes unwinding in preincision | |
| | complex | |
| XPB (ERCC3) | 3′ to 5′ DNA helicase | NM_000122 |
| XPD (ERCC2) | 5′ to 3′ DNA helicase | X52221 |
| GTF2H1 | Core TFIIH subunit p62 | NM_005316 |
| GTF2H2 | Core TFIIH subunit p44 | NM_001515 |
| GTF2H3 | Core TFIIH subunit p34 | NM_001516 |
| GTF2H4 | Core TFIIH subunit p52 | NM_001517 |
| CDK7 | Kinase subunit of TFIIH | NM_001799 |
| CCNH | Kinase subunit of TFIIH | NM_001239 |
| MNAT1 | Kinase subunit of TFIIH | NM_002431 |
| XPG (ERCC5) | 3′ incision | NM_000123 |
| ERCC1 | 5′ incision subunit | NM_001983 |
| XPF (ERCC4) | 5′ incision subunit | NM_005236 |
| LIG1 | DNA joining | NM_000234 |
| NER-related | | |
| CSA (CKN1) | Cockayne syndrome; needed for | NM_000082 |
| | transcription-coupled NER | |
| CSB (ERCC6) | Cockayne syndrome; needed for | NM_000124 |
| | transcription-coupled NER | |
| XAB2 (HCNP) | Cockayne syndrome; needed for | NM_020196 |
| | transcription-coupled NER | |
| DDB1 | Complex defective in XP group E | NM_001923 |
| DDB2 | Mutated in XP group E | NM_000107 |
| MMS19 | Transcription and NER | AW852889 |
| Homologous recombination | | |
| RAD51 | Homologous pairing | NM_002875 |
| RAD51L1 (RAD51B) | Rad51 homolog | U84138 |
| RAD51C | Rad51 homolog | NM_002876 |
| RAD51L3 (RAD51D) | Rad51 homolog | NM_002878 |
| DMC1 | Rad51 homolog, meiosis | NM_007068 |
| XRCC2 | DNA break and cross-link repair | NM_005431 |
| XRCC3 | DNA break and cross-link repair | NM_005432 |
| RAD52 | Accessory factor for recombination | NM_002879 |
| RAD54L | Accessory factor for recombination | NM_003579 |
| RAD54B | Accessory factor for recombination | NM_012415 |
| BRCA1 | Accessory factor for transcription and | NM_007295 |
| | recombination | |
| BRCA2 | Cooperation with RAD51, essential | NM_000059 |
| | function | |
| RAD50 | ATPase in complex with MRE11A, NBS1 | NM_005732 |
| MRE11A | 3′ exonuclease | NM_005590 |
| NBS1 | Mutated in Nijmegen breakage syndrome | NM_002485 |
| Nonhomologous end-joining | | |
| Ku70 (G22P1) | DNA end binding | NM_001469 |
| Ku80 (XRCC5) | DNA end binding | M30938 |
| PRKDC | DNA-dependent protein kinase catalytic | NM_006904 |
| | subunit | |
| LIG4 | Nonhomologous end-joining | NM_002312 |
| XRCC4 | Nonhomologous end-joining | NM_003401 |
| Sanitization of nucleotide pools | | |
| MTH1 (NUDT1) | 8-oxoGTPase | NM_002452 |
| DUT | dUTPase | NM_001948 |
| DNA polymerases (catalytic subunits) | | |
| POLB | BER in nuclear DNA | NM_002690 |
| POLG | BER in mitochondrial DNA | NM_002693 |
| POLD1 | NER and MMR | NM_002691 |
| POLE1 | NER and MMR | NM_006231 |
| PCNA | Sliding clamp for pol delta and pol epsilon | NM_002592 |
| REV3L (POLZ) | DNA pol zeta catalytic subunit, essential | NM_002912 |
| | function | |
| REV7 (MAD2L2) | DNA pol zeta subunit | NM_006341 |
| REV1 | dCMP transferase | NM_016316 |
| POLH | XP variant | NM_006502 |
| POLI (RAD30B) | Lesion bypass | NM_007195 |
| POLQ | DNA cross-link repair | NM_006596 |
| DINB1 (POLK) | Lesion bypass | NM_016218 |
| POLL | Meiotic function | NM_013274 |
| POLM | Presumed specialized lymphoid function | NM_013284 |
| TRF4-1 | Sister-chromatid cohesion | AF089896 |
| TRF4-2 | Sister-chromatid cohesion | AF089897 |
| Editing and processing nucleases | | |
| FEN1 (DNase IV) | 5′ nuclease | NM_004111 |
| TREX1 (DNase III) | 3′ exonuclease | NM_007248 |
| TREX2 | 3′ exonuclease | NM_007205 |
| EX01 (HEX1) | 5′ exonuclease | NM_003686 |
| SPO11 | endonuclease | NM_012444 |
| Rad6 pathway | | |
| UBE2A (RAD6A) | Ubiquitin-conjugating enzyme | NM_003336 |
| UBE2B (RAD6B) | Ubiquitin-conjugating enzyme | NM_003337 |
| RAD18 | Assists repair or replication of damaged | AB035274 |
| | DNA | |
| UBE2VE (MMS2) | Ubiquitin-conjugating complex | AF049140 |
| UBE2N (UBC13, BTG1) | Ubiquitin-conjugating complex | NM_003348 |
| Genes defective in diseases associated | | |
| with sensitivity to DNA damaging agents | | |
| BLM | Bloom syndrome helicase | NM_000057 |
| WRN | Werner syndrome helicase/3′-exonuclease | NM_000553 |
| RECQL4 | Rothmund-Thompson syndrome | NM_004260 |
| ATM | Ataxia telangiectasia | NM_000051 |
| Fanconi anemia | | |
| FANCA | Involved in tolerance or repair of DNA | NM_000135 |
| | cross-links | |
| FANCB | Involved in tolerance or repair of DNA | N/A |
| | cross-links | |
| FANCC | Involved in tolerance or repair of DNA | NM_000136 |
| | cross-links | |
| FANCD | Involved in tolerance or repair of DNA | N/A |
| | cross-links | |
| FANCE | Involved in tolerance or repair of DNA | NM_021922 |
| | cross-links | |
| FANCF | Involved in tolerance or repair of DNA | AF181994 |
| | cross-links | |
| FANCG (XRCC9) | Involved in tolerance or repair of DNA | NM_004629 |
| | cross-links | |
| Other identified genes with a suspected | | |
| DNA repair function | | |
| SNM1 (PS02) | DNA cross-link repair | D42045 |
| SNM1B | Related to SNM1 | AL137856 |
| SNM1C | Related to SNM1 | AA315885 |
| RPA4 | Similar to RPA2 | NM_013347 |
| ABH (ALKB) | Resistance to alkylation damage | X91992 |
| PNKP | Converts some DNA breaks to ligatable | NM_007254 |
| | ends | |
| Other conserved DNA damage response | | |
| genes | | |
| ATR | ATM- and PI-3K-like essential kinase | NM_001184 |
| RAD1 (S. pombe) homolog | PCNA-like DNA damage sensor | NM_002853 |
| RAD9 (S. pombe) homolog | PCNA-like DNA damage sensor | NM_004584 |
| HUS1 (S. pombe) homolog | PCNA-like DNA damage sensor | NM_004507 |
| RAD17 (RAD24) | RFC-like DNA damage sensor | NM_002873 |
| TP53BP1 | BRCT protein | NM_005657 |
| CHEK1 | Effector kinase | NM_001274 |
| CHK2 (Rad53) | Effector kinase | NM_007194 |

TABLE 3

| Gene | | |
|---|---|---|
| Name | Gene Title | Biological Activity |
| RFC2 | replication factor C (activator 1) 2, | DNA replication |
| | 40 kDa | |
| XRCC6 | X-ray repair complementing defective | DNA ligation///DNA repair///double-strand break |
| | repair in Chinese hamster cells 6 (Ku | repair via nonhomologous end-joining///DNA |
| | autoantigen, 70 kDa) | recombination///positive regulation of transcription, |
| | | DNA-dependent///double-strand break repair via |
| | | nonhomologous end-joining///response to DNA damage |
| | | stimulus///DNA recombination |
| APOBEC | apolipoprotein B mRNA editing enzyme, | For all of APOBEC1, APOBEC2, APOBEC3A-H, and |
| | catalytic polypeptide-like | APOBEC4, cytidine deaminases. |
| POLD2 | polymerase (DNA directed), delta 2, | DNA replication///DNA replication |
| | regulatory subunit 50 kDa | |
| PCNA | proliferating cell nuclear antigen | regulation of progression through cell cycle///DNA |
| | | replication///regulation of DNA replication///DNA |
| | | repair///cell proliferation///phosphoinositide-mediated |
| | | signaling///DNA replication |
| RPA1 | replication protein A1, 70 kDa | DNA-dependent DNA replication///DNA repair///DNA |
| | | recombination///DNA replication |
| RPA1 | replication protein A1, 70 kDa | DNA-dependent DNA replication///DNA repair///DNA |
| | | recombination///DNA replication |
| RPA2 | replication protein A2, 32 kDa | DNA replication///DNA-dependent DNA replication |
| ERCC3 | excision repair cross-complementing | DNA topological change///transcription-coupled |
| | rodent repair deficiency, | nucleotide-excision repair///transcription///regulation |
| | complementation group 3 (xeroderma | of transcription, DNA-dependent///transcription from |
| | pigmentosum group B complementing) | RNA polymerase II promoter///induction of apoptosis/// |
| | | sensory perception of sound///DNA repair/// |
| | | nucleotide-excision repair///response to DNA damage |
| | | stimulus///DNA repair |
| UNG | uracil-DNA glycosylase | carbohydrate metabolism///DNA repair///base-excision |
| | | repair///response to DNA damage stimulus///DNA |
| | | repair///DNA repair |
| ERCC5 | excision repair cross-complementing | transcription-coupled nucleotide-excision repair/// |
| | rodent repair deficiency, | nucleotide-excision repair///sensory perception of sound/// |
| | complementation group 5 (xeroderma | DNA repair///response to DNA damage stimulus/// |
| | pigmentosum, complementation group G | nucleotide-excision repair |
| | (Cockayne syndrome)) | |
| MLH1 | mutL homolog 1, colon cancer, | mismatch repair///cell cycle///negative regulation of |
| | nonpolyposis type 2 (E. coli) | progression through cell cycle///DNA repair/// |
| | | mismatch repair///response to DNA damage stimulus |
| LIG1 | ligase I, DNA, ATP-dependent | DNA replication///DNA repair///DNA recombination/// |
| | | cell cycle///morphogenesis///cell division///DNA |
| | | repair///response to DNA damage stimulus///DNA |
| | | metabolism |
| NBN | nibrin | DNA damage checkpoint///cell cycle checkpoint/// |
| | | double-strand break repair |
| NBN | nibrin | DNA damage checkpoint///cell cycle checkpoint/// |
| | | double-strand break repair |
| NBN | nibrin | DNA damage checkpoint///cell cycle checkpoint/// |
| | | double-strand break repair |
| MSH6 | mutS homolog 6 (E. coli) | mismatch repair///DNA metabolism///DNA repair/// |
| | | mismatch repair///response to DNA damage stimulus |
| POLD4 | polymerase (DNA-directed), delta 4 | DNA replication///DNA replication |
| RFC5 | replication factor C (activator 1) 5, | DNA replication///DNA repair///DNA replication |
| | 36.5 kDa | |
| RFC5 | replication factor C (activator 1) 5, | DNA replication///DNA repair///DNA replication |
| | 36.5 kDa | |
| DDB2/// | damage-specific DNA binding protein 2, | nucleotide-excision repair///regulation of transcription, |
| LHX3 | 48 kDa///LIM homeobox 3 | DNA-dependent///organ morphogenesis///DNA repair/// |
| | | response to DNA damage stimulus///DNA repair/// |
| | | transcription///regulation of transcription |
| POLD1 | polymerase (DNA directed), delta 1, | DNA replication///DNA repair///response to UV/// |
| | catalytic subunit 125 kDa | DNA replication |
| FANCG | Fanconi anemia, complementation | cell cycle checkpoint///DNA repair///DNA repair/// |
| | group G | response to DNA damage stimulus///regulation of |
| | | progression through cell cycle |
| POLB | polymerase (DNA directed), beta | DNA-dependent DNA replication///DNA repair///DNA |
| | | replication///DNA repair///response to DNA damage |
| | | stimulus |
| XRCC1 | X-ray repair complementing defective | single strand break repair |
| | repair in Chinese hamster cells 1 | |
| MPG | N-methylpurine-DNA glycosylase | base-excision repair///DNA dealkylation///DNA repair/// |
| | | base-excision repair///response to DNA damage |
| | | stimulus |
| RFC2 | replication factor C (activator 1) 2, | DNA replication |
| | 40 kDa | |
| ERCC1 | excision repair cross-complementing | nucleotide-excision repair///morphogenesis/// |
| | rodent repair deficiency, | nucleotide-excision repair///DNA repair///response to |
| | complementation group 1 (includes | DNA damage stimulus |
| | overlapping antisense sequence) | |
| TDG | thymine-DNA glycosylase | carbohydrate metabolism///base-excision repair///DNA |
| | | repair///response to DNA damage stimulus |
| TDG | thymine-DNA glycosylase | carbohydrate metabolism///base-excision repair///DNA |
| | | repair///response to DNA damage stimulus |
| FANCA | Fanconi anemia, complementation group | DNA repair///protein complex assembly///DNA repair/// |
| | A///Fanconi anemia, complementation | response to DNA damage stimulus |
| | group A | |
| RFC4 | replication factor C (activator 1) 4, | DNA replication///DNA strand elongation///DNA |
| | 37 kDa | repair///phosphoinositide-mediated signaling///DNA |
| | | replication |
| RFC3 | replication factor C (activator 1) 3, | DNA replication///DNA strand elongation |
| | 38 kDa | |
| RFC3 | replication factor C (activator 1) 3, | DNA replication///DNA strand elongation |
| | 38 kDa | |
| APEX2 | APEX nuclease (apurinic/apyrimidinic | DNA repair///response to DNA damage stimulus |
| | endonuclease) 2 | |
| RAD1 | RAD1 homolog (S. pombe) | DNA repair///cell cycle checkpoint///cell cycle |
| | | checkpoint///DNA damage checkpoint///DNA repair/// |
| | | response to DNA damage stimulus///meiotic prophase I |
| RAD1 | RAD1 homolog (S. pombe) | DNA repair///cell cycle checkpoint///cell cycle |
| | | checkpoint///DNA damage checkpoint///DNA repair/// |
| | | response to DNA damage stimulus///meiotic prophase I |
| BRCA1 | breast cancer 1, early onset | regulation of transcription from RNA polymerase II |
| | | promoter///regulation of transcription from RNA |
| | | polymerase III promoter///DNA damage response, |
| | | signal transduction by p53 class mediator resulting in |
| | | transcription of p21 class mediator///cell cycle/// |
| | | protein ubiquitination///androgen receptor signaling |
| | | pathway///regulation of cell proliferation///regulation |
| | | of apoptosis///positive regulation of DNA repair/// |
| | | negative regulation of progression through cell cycle/// |
| | | positive regulation of transcription, DNA-dependent/// |
| | | negative regulation of centriole replication///DNA |
| | | damage response, signal transduction resulting in |
| | | induction of apoptosis///DNA repair///response to |
| | | DNA damage stimulus///protein ubiquitination///DNA |
| | | repair///regulation of DNA repair///apoptosis/// |
| | | response to DNA damage stimulus |
| EXO1 | exonuclease 1 | DNA repair///DNA repair///mismatch repair///DNA |
| | | recombination |
| FEN1 | flap structure-specific endonuclease 1 | DNA replication///double-strand break repair///UV |
| | | protection///phosphoinositide-mediated signaling/// |
| | | DNA repair///DNA replication///DNA repair///DNA |
| | | repair |
| FEN1 | flap structure-specific endonuclease 1 | DNA replication///double-strand break repair///UV |
| | | protection///phosphoinositide-mediated signaling/// |
| | | DNA repair///DNA replication///DNA repair///DNA |
| | | repair |
| MLH3 | mutL homolog 3 (E. coli) | mismatch repair///meiotic recombination///DNA repair/// |
| | | mismatch repair///response to DNA damage stimulus/// |
| | | mismatch repair |
| MGMT | O-6-methylguanine-DNA | DNA ligation///DNA repair///response to DNA |
| | methyltransferase | damage stimulus |
| RAD51 | RAD51 homolog (RecA homolog, E. | double-strand break repair via homologous |
| | coli) (S. cerevisiae) | recombination///DNA unwinding during replication/// |
| | | DNA repair///mitotic recombination///meiosis/// |
| | | meiotic recombination///positive regulation of DNA |
| | | ligation///protein homooligomerization///response to |
| | | DNA damage stimulus///DNA metabolism///DNA |
| | | repair///response to DNA damage stimulus///DNA |
| | | repair///DNA recombination///meiotic recombination/// |
| | | double-strand break repair via homologous |
| | | recombination///DNA unwinding during replication |
| RAD51 | RAD51 homolog (RecA homolog, E. | double-strand break repair via homologous |
| | coli) (S. cerevisiae) | recombination///DNA unwinding during replication/// |
| | | DNA repair///mitotic recombination///meiosis/// |
| | | meiotic recombination///positive regulation of DNA |
| | | ligation///protein homooligomerization///response to |
| | | DNA damage stimulus///DNA metabolism///DNA |
| | | repair///response to DNA damage stimulus///DNA |
| | | repair///DNA recombination///meiotic recombination/// |
| | | double-strand break repair via homologous |
| | | recombination///DNA unwinding during replication |
| XRCC4 | X-ray repair complementing defective | DNA repair///double-strand break repair///DNA |
| | repair in Chinese hamster cells 4 | recombination///DNA recombination///response to |
| | | DNA damage stimulus |
| XRCC4 | X-ray repair complementing defective | DNA repair///double-strand break repair///DNA |
| | repair in Chinese hamster cells 4 | recombination///DNA recombination///response to |
| | | DNA damage stimulus |
| RECQL | RecQ protein-like (DNA helicase Q1- | DNA repair///DNA metabolism |
| | like) | |
| ERCC8 | excision repair cross-complementing | DNA repair///transcription///regulation of |
| | rodent repair deficiency, | transcription, DNA-dependent///sensory perception of |
| | complementation group 8 | sound///transcription-coupled nucleotide-excision repair |
| FANCC | Fanconi anemia, complementation group | DNA repair///DNA repair///protein complex assembly/// |
| | C | response to DNA damage stimulus |
| OGG1 | 8-oxoguanine DNA glycosylase | carbohydrate metabolism///base-excision repair///DNA |
| | | repair///base-excision repair///response to DNA |
| | | damage stimulus///DNA repair |
| MRE11A | MRE11 meiotic recombination 11 | regulation of mitotic recombination///double-strand |
| | homolog A (S. cerevisiae) | break repair via nonhomologous end-joining/// |
| | | telomerase-dependent telomere maintenance///meiosis/// |
| | | meiotic recombination///DNA metabolism///DNA |
| | | repair///double-strand break repair///response to DNA |
| | | damage stimulus///DNA repair///double-strand break |
| | | repair///DNA recombination |
| RAD52 | RAD52 homolog (S. cerevisiae) | double-strand break repair///mitotic recombination/// |
| | | meiotic recombination///DNA repair///DNA |
| | | recombination///response to DNA damage stimulus |
| WRN | Werner syndrome | DNA metabolism///aging |
| XPA | xeroderma pigmentosum, | nucleotide-excision repair///DNA repair///response to |
| | complementation group A | DNA damage stimulus///DNA repair///nucleotide- |
| | | excision repair |
| BLM | Bloom syndrome | DNA replication///DNA repair///DNA recombination/// |
| | | antimicrobial humoral response (sensu Vertebrata)/// |
| | | DNA metabolism///DNA replication |
| OGG1 | 8-oxoguanine DNA glycosylase | carbohydrate metabolism///base-excision repair///DNA |
| | | repair///base-excision repair///response to DNA |
| | | damage stimulus///DNA repair |
| MSH3 | mutS homolog 3 (E. coli) | mismatch repair///DNA metabolism///DNA repair/// |
| | | mismatch repair///response to DNA damage stimulus |
| POLE2 | polymerase (DNA directed), epsilon 2 | DNA replication///DNA repair///DNA replication |
| | (p59 subunit) | |
| RAD51C | RAD51 homolog C (S. cerevisiae) | DNA repair///DNA recombination///DNA metabolism/// |
| | | DNA repair///DNA recombination///response to |
| | | DNA damage stimulus |
| LIG4 | ligase IV, DNA, ATP-dependent | single strand break repair///DNA replication///DNA |
| | | recombination///cell cycle///cell division///DNA |
| | | repair///response to DNA damage stimulus |
| ERCC6 | excision repair cross-complementing | DNA repair///transcription///regulation of |
| | rodent repair deficiency, | transcription, DNA-dependent///transcription from RNA |
| | complementation group 6 | polymerase II promoter///sensory perception of sound |
| LIG3 | ligase III, DNA, ATP-dependent | DNA replication///DNA repair///cell cycle///meiotic |
| | | recombination///spermatogenesis///cell division/// |
| | | DNA repair///DNA recombination///response to DNA |
| | | damage stimulus |
| RAD17 | RAD17 homolog (S. pombe) | DNA replication///DNA repair///cell cycle///response |
| | | to DNA damage stimulus |
| XRCC2 | X-ray repair complementing defective | DNA repair///DNA recombination///meiosis///DNA |
| | repair in Chinese hamster cells 2 | metabolism///DNA repair///response to DNA damage |
| | | stimulus |
| MUTYH | mutY homolog (E. coli) | carbohydrate metabolism///base-excision repair/// |
| | | mismatch repair///cell cycle///negative regulation of |
| | | progression through cell cycle///DNA repair///response |
| | | to DNA damage stimulus///DNA repair |
| RFC1 | replication factor C (activator 1) 1, | DNA-dependent DNA replication///transcription/// |
| | 145 kDa///replication factor C (activator | regulation of transcription, DNA-dependent/// |
| | 1) 1, 145 kDa | telomerase-dependent telomere maintenance///DNA |
| | | replication///DNA repair |
| RFC1 | replication factor C (activator 1) 1, | DNA-dependent DNA replication///transcription/// |
| | 145 kDa | regulation of transcription, DNA-dependent/// |
| | | telomerase-dependent telomere maintenance///DNA |
| | | replication///DNA repair |
| BRCA2 | breast cancer 2, early onset | regulation of progression through cell cycle///double- |
| | | strand break repair via homologous recombination/// |
| | | DNA repair///establishment and/or maintenance of |
| | | chromatin architecture///chromatin remodeling/// |
| | | regulation of S phase of mitotic cell cycle///mitotic |
| | | checkpoint///regulation of transcription///response to |
| | | DNA damage stimulus |
| RAD50 | RAD50 homolog (S. cerevisiae) | regulation of mitotic recombination///double-strand |
| | | break repair///telomerase-dependent telomere |
| | | maintenance///cell cycle///meiosis///meiotic |
| | | recombination///chromosome organization and |
| | | biogenesis///telomere maintenance///DNA repair/// |
| | | response to DNA damage stimulus///DNA repair/// |
| | | DNA recombination |
| DDB1 | damage-specific DNA binding protein 1, | nucleotide-excision repair///ubiquitin cycle///DNA |
| | 127 kDa | repair///response to DNA damage stimulus///DNA |
| | | repair |
| XRCC5 | X-ray repair complementing defective | double-strand break repair via nonhomologous end- |
| | repair in Chinese hamster cells 5 | joining///DNA recombination///DNA repair///DNA |
| | (double-strand-break rejoining; Ku | recombination///response to DNA damage stimulus/// |
| | autoantigen, 80 kDa) | double-strand break repair |
| XRCC5 | X-ray repair complementing defective | double-strand break repair via nonhomologous end- |
| | repair in Chinese hamster cells 5 | joining///DNA recombination///DNA repair///DNA |
| | (double-strand-break rejoining; Ku | recombination///response to DNA damage stimulus/// |
| | autoantigen, 80 kDa) | double-strand break repair |
| PARP1 | poly (ADP-ribose) polymerase family, | DNA repair///transcription from RNA polymerase II |
| | member 1 | promoter///protein amino acid ADP-ribosylation/// |
| | | DNA metabolism///DNA repair///protein amino acid |
| | | ADP-ribosylation///response to DNA damage stimulus |
| POLE3 | polymerase (DNA directed), epsilon 3 | DNA replication |
| | (p17 subunit) | |
| RFC1 | replication factor C (activator 1) 1, | DNA-dependent DNA replication///transcription/// |
| | 145 kDa | regulation of transcription, DNA-dependent/// |
| | | telomerase-dependent telomere maintenance///DNA |
| | | replication///DNA repair |
| RAD50 | RAD50 homolog (S. cerevisiae) | regulation of mitotic recombination///double-strand |
| | | break repair///telomerase-dependent telomere |
| | | maintenance///cell cycle///meiosis///meiotic |
| | | recombination///chromosome organization and |
| | | biogenesis///telomere maintenance///DNA repair/// |
| | | response to DNA damage stimulus///DNA repair/// |
| | | DNA recombination |
| XPC | xeroderma pigmentosum, | nucleotide-excision repair///DNA repair///nucleotide- |
| | complementation group C | excision repair///response to DNA damage stimulus/// |
| | | DNA repair |
| MSH2 | mutS homolog 2, colon cancer, | mismatch repair///postreplication repair///cell cycle/// |
| | nonpolyposis type 1 (E. coli) | negative regulation of progression through cell cycle/// |
| | | DNA metabolism///DNA repair///mismatch repair/// |
| | | response to DNA damage stimulus///DNA repair |
| RPA3 | replication protein A3, 14 kDa | DNA replication///DNA repair///DNA replication |
| MBD4 | methyl-CpG binding domain protein 4 | base-excision repair///DNA repair///response to DNA |
| | | damage stimulus///DNA repair |
| MBD4 | methyl-CpG binding domain protein 4 | base-excision repair///DNA repair///response to DNA |
| | | damage stimulus///DNA repair |
| NTHL1 | nth endonuclease III-like 1 (E. coli) | carbohydrate metabolism///base-excision repair/// |
| | | nucleotide-excision repair, DNA incision, 5′-to lesion/// |
| | | DNA repair///response to DNA damage stimulus |
| PMS2/// | PMS2 postmeiotic segregation increased | mismatch repair///cell cycle///negative regulation of |
| PMS2CL | 2 (S. cerevisiae)///PMS2-C terminal-like | progression through cell cycle///DNA repair/// |
| | | mismatch repair///response to DNA damage stimulus/// |
| | | mismatch repair |
| RAD51C | RAD51 homolog C (S. cerevisiae) | DNA repair///DNA recombination///DNA metabolism/// |
| | | DNA repair///DNA recombination///response to |
| | | DNA damage stimulus |
| UNG2 | uracil-DNA glycosylase 2 | regulation of progression through cell cycle/// |
| | | carbohydrate metabolism///base-excision repair///DNA |
| | | repair///response to DNA damage stimulus |
| APEX1 | APEX nuclease (multifunctional DNA | base-excision repair///transcription from RNA |
| | repair enzyme) 1 | polymerase II promoter///regulation of DNA binding/// |
| | | DNA repair///response to DNA damage stimulus |
| ERCC4 | excision repair cross-complementing | nucleotide-excision repair///nucleotide-excision repair/// |
| | rodent repair deficiency, | DNA metabolism///DNA repair///response to DNA |
| | complementation group 4 | damage stimulus |
| RAD1 | RAD1 homolog (S. pombe) | DNA repair///cell cycle checkpoint///cell cycle |
| | | checkpoint///DNA damage checkpoint///DNA repair/// |
| | | response to DNA damage stimulus///meiotic prophase I |
| RECQL5 | RecQ protein-like 5 | DNA repair///DNA metabolism///DNA metabolism |
| MSH5 | mutS homolog 5 (E. coli) | DNA metabolism///mismatch repair///mismatch repair/// |
| | | meiosis///meiotic recombination///meiotic prophase |
| | | II///meiosis |
| RECQL | RecQ protein-like (DNA helicase Q1- | DNA repair///DNA metabolism |
| | like) | |
| RAD52 | RAD52 homolog (S. cerevisiae) | double-strand break repair///mitotic recombination/// |
| | | meiotic recombination///DNA repair///DNA |
| | | recombination///response to DNA damage stimulus |
| XRCC4 | X-ray repair complementing defective | DNA repair///double-strand break repair///DNA |
| | repair in Chinese hamster cells 4 | recombination///DNA recombination///response to |
| | | DNA damage stimulus |
| XRCC4 | X-ray repair complementing defective | DNA repair///double-strand break repair///DNA |
| | repair in Chinese hamster cells 4 | recombination///DNA recombination///response to |
| | | DNA damage stimulus |
| RAD17 | RAD17 homolog (S. pombe) | DNA replication///DNA repair///cell cycle///response |
| | | to DNA damage stimulus |
| MSH3 | mutS homolog 3 (E. coli) | mismatch repair///DNA metabolism///DNA repair/// |
| | | mismatch repair///response to DNA damage stimulus |
| MRE11A | MRE11 meiotic recombination 11 | regulation of mitotic recombination///double-strand |
| | homolog A (S. cerevisiae) | break repair via nonhomologous end-joining/// |
| | | telomerase-dependent telomere maintenance///meiosis/// |
| | | meiotic recombination///DNA metabolism///DNA |
| | | repair///double-strand break repair///response to DNA |
| | | damage stimulus///DNA repair///double-strand break |
| | | repair///DNA recombination |
| MSH6 | mutS homolog 6 (E. coli) | mismatch repair///DNA metabolism///DNA repair/// |
| | | mismatch repair///response to DNA damage stimulus |
| MSH6 | mutS homolog 6 (E. coli) | mismatch repair///DNA metabolism///DNA repair/// |
| | | mismatch repair///response to DNA damage stimulus |
| RECQL5 | RecQ protein-like 5 | DNA repair///DNA metabolism///DNA metabolism |
| BRCA1 | breast cancer 1, early onset | regulation of transcription from RNA polymerase II |
| | | promoter///regulation of transcription from RNA |
| | | polymerase III promoter///DNA damage response, |
| | | signal transduction by p53 class mediator resulting in |
| | | transcription of p21 class mediator///cell cycle/// |
| | | protein ubiquitination///androgen receptor signaling |
| | | pathway///regulation of cell proliferation///regulation |
| | | of apoptosis///positive regulation of DNA repair/// |
| | | negative regulation of progression through cell cycle/// |
| | | positive regulation of transcription, DNA-dependent/// |
| | | negative regulation of centriole replication///DNA |
| | | damage response, signal transduction resulting in |
| | | induction of apoptosis///DNA repair///response to |
| | | DNA damage stimulus///protein ubiquitination///DNA |
| | | repair///regulation of DNA repair///apoptosis/// |
| | | response to DNA damage stimulus |
| RAD52 | RAD52 homolog (S. cerevisiae) | double-strand break repair///mitotic recombination/// |
| | | meiotic recombination///DNA repair///DNA |
| | | recombination///response to DNA damage stimulus |
| POLD3 | polymerase (DNA-directed), delta 3, | DNA synthesis during DNA repair///mismatch repair/// |
| | accessory subunit | DNA replication |
| MSH5 | mutS homolog 5 (E. coli) | DNA metabolism///mismatch repair///mismatch repair/// |
| | | meiosis///meiotic recombination///meiotic prophase |
| | | II///meiosis |
| ERCC2 | excision repair cross-complementing | transcription-coupled nucleotide-excision repair/// |
| | rodent repair deficiency, | transcription///regulation of transcription, DNA- |
| | complementation group 2 (xeroderma | dependent///transcription from RNA polymerase II |
| | pigmentosum D) | promoter///induction of apoptosis///sensory perception |
| | | of sound///nucleobase, nucleoside, nucleotide and |
| | | nucleic acid metabolism///nucleotide-excision repair |
| RECQL4 | RecQ protein-like 4 | DNA repair///development///DNA metabolism |
| PMS1 | PMS1 postmeiotic segregation increased | mismatch repair///regulation of transcription, DNA- |
| | 1 (S. cerevisiae) | dependent///cell cycle///negative regulation of |
| | | progression through cell cycle///mismatch repair/// |
| | | DNA repair///response to DNA damage stimulus |
| ZFP276 | zinc finger protein 276 homolog (mouse) | transcription///regulation of transcription, DNA- |
| | | dependent |
| MBD4 | methyl-CpG binding domain protein 4 | base-excision repair///DNA repair///response to DNA |
| | | damage stimulus///DNA repair |
| MBD4 | methyl-CpG binding domain protein 4 | base-excision repair///DNA repair///response to DNA |
| | | damage stimulus///DNA repair |
| MLH3 | mutL homolog 3 (E. coli) | mismatch repair///meiotic recombination///DNA repair/// |
| | | mismatch repair///response to DNA damage stimulus/// |
| | | mismatch repair |
| FANCA | Fanconi anemia, complementation group | DNA repair///protein complex assembly///DNA repair/// |
| | A | response to DNA damage stimulus |
| POLE | polymerase (DNA directed), epsilon | DNA replication///DNA repair///DNA replication/// |
| | | response to DNA damage stimulus |
| XRCC3 | X-ray repair complementing defective | DNA repair///DNA recombination///DNA metabolism/// |
| | repair in Chinese hamster cells 3 | DNA repair///DNA recombination///response to |
| | | DNA damage stimulus///response to DNA damage |
| | | stimulus |
| MLH3 | mutL homolog 3 (E. coli) | mismatch repair///meiotic recombination///DNA repair/// |
| | | mismatch repair///response to DNA damage stimulus/// |
| | | mismatch repair |
| NBN | nibrin | DNA damage checkpoint///cell cycle checkpoint/// |
| | | double-strand break repair |
| SMUG1 | single-strand selective monofunctional | carbohydrate metabolism///DNA repair///response to |
| | uracil DNA | glycosylase DNA damage stimulus |
| FANCF | Fanconi anemia, complementation group | DNA repair///response to DNA damage stimulus |
| | F | |
| NEIL1 | nei endonuclease VIII-like 1 (E. coli) | carbohydrate metabolism///DNA repair///response to |
| | | DNA damage stimulus |
| FANCE | Fanconi anemia, complementation group | DNA repair///response to DNA damage stimulus |
| | E | |
| MSH5 | mutS homolog 5 (E. coli) | DNA metabolism///mismatch repair///mismatch repair/// |
| | | meiosis///meiotic recombination///meiotic prophase |
| | | II///meiosis |
| RECQL5 | RecQ protein-like 5 | DNA repair///DNA metabolism///DNA metabolism |

Therefore, it should be appreciated that any one or more of the above genes in Tables 1-3 can be assessed for mutations (which may be further classified or assessed into mutations affecting function or silent mutations), for copy number, and/or for expression strength, as well as RNA splice variants and differences in polyadenylation or other parameters that affect stability or half-life of a transcript. Likewise, protein quantity and/or protein activities for the corresponding proteins encoded by the genes of Tables 1-3 may be determined using mass spec or in vitro assays well known in the art. Consequently, the repair status of a cell can be assessed using the omics data across a wide variety of repair mechanisms. As such, one or more deficiencies (functional and/or by decreased quantity) in DNA repair genes relative to normal may be indicative of a diseased cell or lack of repair capability, which in turn may be indicative for treatment success using DNA damaging agents. On the other hand, over-activity or overexpression (relative to a healthy cell of the same individual) of one or more DNA repair genes may be indicative of DNA damage, presence or exposure to a DNA damaging environment or agent. Moreover, functional defects in DNA repair genes may be indicative of a predisposition to hypermutations.

As will be also readily appreciated, the DNA repair function as assessed by omics data can be correlated with damage patterns that are present or that can be expected. Thus, analysis of mutation signatures (see e.g., URL:cancer.sanger.ac.uk/cosmic/signatures) in conjunction with the teachings presented herein is also contemplated. For example, mutation signatures 2 and 13 have been attributed to activity of the AID/APOBEC family of cytidine deaminases, while signature 4 exhibits transcriptional strand bias for C>A mutations, which is compatible with the notion that damage to guanine is repaired by transcription-coupled nucleotide excision repair. Mutation signature 26 is associated with defective DNA mismatch repair. Most typically, the observed or expected mutation signatures will generally correlate with a reduced or increased activity of DNA corresponding repair genes, the type of tumor, and/or exposure to DNA damaging agents (environmental, or drug-associated).

Of course, it should be appreciated that analyses presented herein may be performed over specific and diverse populations to thereby obtain reference values for the specific populations, such as across various health associated states (e.g., healthy, diagnosed with a specific disease and/or disease state, which may or may not be inherited, or which may or may not be associated with impaired DNA repair), a specific age or age bracket, a specific ethnic group that may or may not be associated with longevity or high morbidity/mortality (e.g., Okinawa Japanese, Nepalese, Sri Lankans, etc.), and/or pharmaceutical treatment (e.g., treatment with DNA alkylating agents, DNA crosslinkers, DNA intercalators, or platinum adducts). Of course, populations may also be enlisted from databases with known omics information, and especially publically available omics information from cancer patients (e.g., TCGA, COSMIC, etc.) and proprietary databases from a large variety of individuals that may be healthy or diagnosed with a disease. Likewise, it should be appreciated that the population records may also be indexed over time for the same individual or group of individuals, which advantageously allows detection of shifts or changes in the genes and pathways associated with RNA repair.

Thus, it should be recognized that contemplated systems and methods allow for a large cross sectional database for DNA repair gene activity, which in turn allows the generation of a risk matrix that may be based on individual DNA repair gene scores, on ratio scores, sum scores, differential scores, etc. In particularly preferred aspects, it is contemplated that an error score can be established for one or more DNA repair genes, and that the score may be reflective of or even prognostic for various diseases that are at least in part due to mutations in DNA repair genes and/or pathways. For example, especially suitable error scores may involve scores for one or more genes associated with one or more types of DNA repair (e.g., base excision repair, homologous recombination repair, etc.) relative to another gene that may or may not be associated with one type of DNA repair (e.g., TP53, Fas, bcl-2, CHK2, Non-homologous end-joining repair gene, etc.). In another example, contemplated error scores may involve scores for one or more genes associated with one or more types of DNA repair (e.g., base excision repair, homologous recombination repair, etc.) relative to an overall mutation rate to so better identify DNA repair relevant mutations over ‘background’ mutations. In still other examples, mutations in some DNA repair genes may be ‘leading indicators’ or triggers to activate other DNA repair mechanism such as p53 mediated repair. Identification of such triggers may advantageously allow for early diagnosis of repair events, or may be used to trigger repair events.

Based on the particular quantitation and/or analysis of the omics data, it should be noted that various calculations can be performed. For example, the omics data may be used to generate a general error status for an individual (or tumor within an individual), or to associate the number and/or type of alterations in DNA repair genes to identify a ‘tipping point’ for one or more DNA repair gene mutations after which a general mutation rate skyrockets. For example, where a rate or number of mutations in ERCC1 and other DNA repair genes could have only minor systemic consequence, addition of further mutations to TP53 may result in a catastrophic increase in mutation rates. Thus, and viewed from a different perspective, mutations in the genes associated with DNA may be used to estimate the risk of occurrence for a DNA damage-based disease, and especially cancer and age-related diseases. In still further contemplated uses, so obtained omics information may be analyzed in one or more pathway analysis algorithms (e.g., PARADIGM) to so identify affected pathways and to so possibly adjust treatment where treatment employs DNA damaging agents. Pathway analysis algorithms may also be used to in silico modulate expression of one or more DNA repair genes, which may results in desirable or even unexpected in silico treatment outcomes, which may be translated into the clinic. Likewise, various machine learning algorithms may be employed to associate a disease parameter (e.g., type of disease, stage of disease, treatability of a disease with specific drug) with the omics data for the genes associated with DNA repair) to so identify a specific mutation pattern as being correlated with a particular condition or drug sensitivity.

In still further contemplated aspects, it should be appreciated that once one or more genes associated with DNA repair have been identified as dysfunctional (e.g., over-expressed, under-expressed, mutated, truncated, splice variant present, etc.), drugs can be identified to counteract the dysfunctional gene. As noted above, such drugs can be identified using large small molecule libraries, computational approaches, and/or data from the public domain. Moreover, in silico simulations using pathway models may be employed to identify such drugs. Consequently, it should be appreciated that contemplated system and methods may not only be of diagnostic value, but also be employed to identify and use drugs that counteract mutation-related diseases, and especially cancer and age-related diseases. In such systems, one or more drugs can then be administered to an individual to counteract DNA repair activity, and/or to treat a specific cell population that is characterized by a DNA repair signature.

Therefore, contemplated omics analyses are also particularly useful for monitoring treatment of a patient that is subject to a pharmaceutical intervention. Such monitoring will advantageously include detection and/or quantification of diseased cells having a specific repair signature, detection of triggering DNA repair in healthy tissue during treatment with DNA damaging agents, detection of development of treatment resistant clonal populations having a specific repair signature, and detection of disease recurrence where the diseased cells have a particular repair signature. Viewed from a different perspective, the signatures may also be used to identify whether or not a cell population is likely sensitive to treatment with DNA damaging agents. Similarly, the signatures may also be used in a combination treatment where an individual receives treatment with a DNA damaging agent and at the same time one or more pharmaceutical agents that inhibit the corresponding DNA repair genes required to repair the damage brought on by the DNA damaging agent. Such strategy may be readily monitored using contemplated omics tests. Thus, and viewed from yet another perspective, contemplated methods may be employed to specifically identify and then target DNA repair mechanisms (e.g., using PARP inhibitors, Chk1-2 inhibitors, WEE-1 inhibitors, or ATR inhibitors) that may be used by a cell to counteract treatment with a DNA damaging agent.

### EXAMPLE 1

A whole blood sample is provided and divided into two aliquots. A first aliquot is used to isolate cell free RNA, cfRNA (and where desired cell free DNA, cfDNA) as described below. However, various other bodily fluids are also deemed appropriate so long as cfRNA is present in such fluids. Appropriate fluids include saliva, ascites fluid, spinal fluid, urine, etc, which may be fresh, chemically preserved, or refrigerated or frozen. For example, specimens can be accepted as 10 ml of whole blood drawn into commercially available cell-free RNA BCT® tubes or cell-free DNA BCT® tubes (Streck, 7002 S. 109 St., Omaha, Nebr. 68128) containing RNA or DNA stabilizers, respectively. Advantageously, cfRNA is stable in whole blood in the cell-free RNA BCT tubes for seven days while cfDNA is stable in whole blood in the cell-free DNA BCT Tubes for fourteen days, allowing time for shipping of patient samples from world-wide locations without the degradation of cfRNA or cfDNA. Moreover, it is generally preferred that the cfRNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, RNA stabilization reagents will not lead to a substantial increase (e.g., increase in total RNA no more than 10%, or no more than 5%, or no more than 2%, or no more than 1%) in RNA quantities in serum or plasma after the reagents are combined with blood.

Most typically, but not necessarily, the first aliquot is centrifuged in the presence of an RNase inhibitor, a preservative agent, a metabolic inhibitor, and a chelator. Moreover, it is generally preferred that the step of centrifuging whole blood is performed under conditions that preserve the integrity of cellular components. For example, the first RCF may be between 700 and 2,500 (e.g., 1,600), and/or the second RCF may be between 7,000 and 25,000 (e.g., 16,000), wherein centrifugation at the first RCF is performed between 15-25 minutes (e.g., 20 minutes) and wherein the centrifugation at the second RCF is performed between 5-15 minutes (e.g., 10 minutes). Where desired or required, cfRNA may be stored at −80° C. and/or cDNA prepared from the cfRNA may be stored at −4° C.

A second aliquot of the whole blood sample can be centrifuged in an evacuated blood collection tube to separate the cells from the serum/plasma. Once isolated, the cells can be washed in isotonic ringer solution and then lysed to so prepare DNA and RNA using one or more commercially available test kits (e.g., Qiagen DNA blood mini kit, Qiagen RNA blood mini kit).

For both analyses, DNA and RNA sequencing is performed. In addition, quantitative RNA analysis is employed to obtain transcriptomics information. Where available, proteomics analysis is performed using selected reaction monitoring for at least two, or at least 4, or at least 10, or at least 20 different proteins associated with DNA repair. So obtained omics information can then be processed using pathway analysis (especially using PARADIGM) to identify any impact of any mutations on DNA repair pathways.

### EXAMPLE 2

A whole blood sample is drawn from a patient diagnosed with cancer and processed as noted in Example 1 above. In addition, a fresh tumor biopsy is obtained and a full omics analysis performed in which DNA sequencing is whole genome sequencing at a depth of at least 20× for DNA and RNA. In addition, quantitative RNA analysis is employed to obtain transcriptomics information. Where available, proteomics analysis is performed using selected reaction monitoring for at least two, or at least 4, or at least 10, or at least 20 different proteins associated with DNA repair. Where desired, proteomics analysis is performed using selected reaction monitoring for at least two, or at least 4, or at least 10, or at least 20 different proteins associated with DNA repair. So obtained omics information can then be processed using pathway analysis (especially using PARADIGM) to identify any impact of any mutations on DNA repair pathways.

### EXAMPLE 3

Once omics analysis for a patient sample (e.g., of Example 2) is concluded, changes in DNA, RNA, and protein (activities) relative to omics data of age-matched healthy individuals are noted. Such changes may be labeled idiosyncratic where no statistical association with a known disease pattern is observed, or changes may be associated with a pattern that is characteristic of a disease. As noted above, analysis may include observation on individual genes associated with DNA repair, or on multiple genes, alone or in various relationships (e.g., ratio, sum, etc.).

### EXAMPLE 4

A tumor biopsy and a biopsy of corresponding non-tumor tissue (or a blood sample) is obtained from an individual. The tumor biopsy is then subjected to DNA sequencing and RNAseq with quantification of expressed RNA in the tumor cells. Mutational status for all DNA repair genes is determined as well as the transcription strength, for both the biopsy sample and the corresponding non-tumor tissue. Differences in repair status are ascertained and treatment with DNA damaging agents (e.g., using crosslinkers, intercalating agents, etc.) is started. Treatment is then monitored either by re-biopsy of the tumor or by isolation and analysis of cfDNA and cfRNA for DNA repair genes as discussed above. Where an increase in DNA repair gene expression is noted in the tumor sample, inhibitors for DNA repair may be administered. Moreover, pathway analysis (e.g., using PARADIGM) can be performed using the omics data to identify further treatment options that will selectively interfere with tumor DNA repair. During such follow-up, repair signatures may be obtained for the tumor to identify clonal development, evolution of resistance, and or tumor status. Upon conclusion, repair signatures may be obtained (typically from cell free DNA and cell free RNA to detect tumor specific repair signatures, which may be indicative of recurrence.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of analyzing omics data, comprising:
obtaining omics data for a plurality of DNA damage repair genes, wherein the omics data comprise at least two of DNA sequence data, RNA sequence data, transcription strength, and protein activity or quantity; and
associating the omics data with at least one of a health status, an omics error status, age, a disease, a prophylactic recommendation, and a therapeutic recommendation.

2. The method of claim 1 further comprising a step of calculating a score from the omics data to so obtain a health score.

3. The method of any one of the preceding claims wherein the DNA sequence data are selected from the group consisting of mutation data, copy number data duplication, loss of heterozygosity data, and epigenetic status.

4. The method of any one of the preceding claims wherein the RNA sequence data are selected from the group consisting of mRNA sequence data and splice variant data.

5. The method of any one of the preceding claims wherein the RNA sequence data are obtained from the group consisting of RNA from solid tissue, RNA from blood cells, and circulating cell free RNA.

6. The method of any one of the preceding claims wherein the transcription strength is expressed as transcripts of the damage repair gene per million transcripts.

7. The method of any one of the preceding claims wherein the protein activity or quantity is determined using a mass spectroscopic method.

8. The method of any one of the preceding claims wherein the health status is selected from the group consisting of healthy, diagnosed with an age related disease, and diagnosed with cancer.

9. The method of any one of the preceding claims wherein the prophylactic recommendation comprises a recommendation to treat an individual with an agent that modulates expression of at least one of the plurality of DNA damage repair genes.

10. The method of any one of the preceding claims wherein the therapeutic recommendation comprises a recommendation to treat a patient with a DNA damaging agent.

11. The method of any one of the preceding claims wherein the plurality of DNA damage repair genes is selected from at least one of a base excision repair gene, a mismatch repair gene, a nucleotide excision repair gene, a homologous recombination gene, and a non-homologous end-joining gene.

12. The method of any one of the preceding claims wherein the plurality of DNA damage repair genes is selected from at least two of a base excision repair gene, a mismatch repair gene, a nucleotide excision repair gene, a homologous recombination gene, and a non-homologous end-joining gene.

13. The method of any one of the preceding claims wherein the plurality of DNA damage repair genes is selected from at least three of a base excision repair gene, a mismatch repair gene, a nucleotide excision repair gene, a homologous recombination gene, and a non-homologous end-joining gene.

14. The method of any one of the preceding claims wherein the plurality of DNA damage repair genes is selected from a base excision repair gene, a mismatch repair gene, a nucleotide excision repair gene, a homologous recombination gene, and a non-homologous end-joining gene.

15. The method of any one of the preceding claims wherein the plurality of DNA damage repair genes are at least two genes selected from the genes listed in Table 1, Table 2, and Table 3.

16. The method of any one of the preceding claims wherein the step of associating comprises a weight score for at least one of the omics data.

17. The method of any one of the preceding claims further comprising a step of comparing the omics error status with a threshold value to thereby determine a risk score. (tipping point')

18. A method of calculating a health indicator, comprising:
obtaining omics data for a plurality of DNA damage repair genes, wherein the omics data comprise at least two of DNA sequence data, RNA sequence data, transcription strength, and protein activity or quantity; and
using the omics data for the plurality of DNA damage repair genes to generate a health compound score that is indicative of the health of a person.

19. The method of claim 18 further comprising a step of comparing the compound score with a threshold value to thereby determine a treatment option.

20. The method of claim 19 wherein the treatment option is a prophylactic treatment where the compound score is below the threshold value.

21. The method of claim 19 wherein the treatment option uses a drug that modulates expression of at least one of the plurality of DNA damage repair genes.

22. The method of claim 19 wherein the treatment option uses a drug that induces DNA damage.

23. A method of treating an individual, comprising:
obtaining omics data for a plurality of DNA damage repair genes, wherein the omics data comprise at least two of DNA sequence data, RNA sequence data, transcription strength, and protein activity or quantity;
identifying at least one of the DNA damage repair genes as being dysregulated relative to a corresponding healthy control; and
administering an agent that counteracts the at least one of the dysregulated DNA damage repair gene.

24. The method of claim 23 wherein the DNA sequence data are selected from the group consisting of mutation data, copy number data duplication, loss of heterozygosity data, and epigenetic status.

25. The method of any one of claims 23-24 wherein the RNA sequence data are selected from the group consisting of mRNA sequence data and splice variant data.

26. The method of any one of claims 23-25 wherein the RNA sequence data are obtained from the group consisting of RNA from solid tissue, RNA from blood cells, and circulating cell free RNA.

27. The method of any one of claims 23-26 wherein the transcription strength is expressed as transcripts of the damage repair gene per million transcripts.

28. The method of any one of claims 23-27 wherein the protein activity or quantity is determined using a mass spectroscopic method.

29. The method of any one of claims 23-28 wherein the at least one of the DNA damage repair gene is selected from a base excision repair gene, a mismatch repair gene, a nucleotide excision repair gene, a homologous recombination gene, and a non-homologous end-joining gene.

30. The method of any one of claims 23-28 wherein the plurality of DNA damage repair genes are at least two genes selected from the genes listed in Table 1, Table 2, and Table 3.

31. A method of performing a test on a subject, comprising:
obtaining a blood sample from the subject;
using the blood sample to obtain omics data for a plurality of DNA damage repair genes, wherein the omics data comprise at least two of DNA sequence data, RNA sequence data, transcription strength, and protein activity or quantity;
wherein the omics data are obtained from at least one of a cell free portion of the blood sample and a cell containing portion of the blood sample;
identifying at least one of the DNA damage repair genes in the blood sample as being dysregulated relative to a corresponding healthy control.

32. The method of claim 31 wherein the omics data are obtained from the cell free portion of the blood sample.

33. The method of any one of claims 31-32 wherein the RNA sequence data are selected from the group consisting of mRNA sequence data and splice variant data.

34. The method of any one of claims 31-33 wherein the RNA sequence data are obtained from the group consisting of RNA from solid tissue, RNA from blood cells, and circulating cell free RNA.

35. The method of any one of claims 31-34 wherein the transcription strength is expressed as transcripts of the damage repair gene per million transcripts.

36. The method of any one of claims 31-35 wherein the at least one of the DNA damage repair gene is selected from a base excision repair gene, a mismatch repair gene, a nucleotide excision repair gene, a homologous recombination gene, and a non-homologous end-joining gene.

37. The method of any one of claims 31-35 wherein the plurality of DNA damage repair genes are at least two genes selected from the genes listed in Table 1, Table 2, and Table 3.
